(19) United States
(12) Patent Application Publication
(10) Pub. No.: US 2006/0069375 A1
(43) Pub. Date: Mar. 30, 2006

(54) Absorbent article with C-folded moisture barrier and methods of manufacturing same

(76) Inventors: Andrew Waksmundzki, Jackson, NJ (US); John Litvay, Downingtown, PA (US); Frank Glaug, Chester Springs, PA (US)

Correspondence Address: Christopher C. Campbell, Esq.;Hunton & Williams, Suite 1200, 1900 K Street, NW, Washington, DC, 20006-1109, US

(21) Appl. No.: 10/951,889

(22) Filed: Sep. 29, 2004

(51) Int. Cl. A61F 13/15

(52) U.S. Cl. 604/385.201

(57) ABSTRACT

An absorbent core and method of manufacturing an absorbent core for use with various absorbent articles comprising a top sheet layer, a back sheet layer and an absorbent core located between at least portions of the top sheet layer and back sheet layer. The absorbent core incorporates a C-shaped fluid barrier material surrounding at least the back sheet facing surface and side surfaces of the absorbent core. One or more fluid acquisition layers may reside over the absorbent core between the core and the top sheet layer. One or more fluid management layers may also reside within the core.

### FIELD OF THE INVENTION

The present invention relates to absorbent articles and methods of manufacturing absorbent articles and more particularly to absorbent articles and methods of manufacturing absorbent articles having an improved fluid retention property through the use of a C-folded moisture barrier surrounding the absorbent core.

### BACKGROUND OF THE INVENTION

Disposable absorbent articles, including child diapers, training pants, adult incontinence garments, and feminine napkin products are known in the art. Though different in appearance and dimensions, all of these products perform the same basic function of fluid absorption and retention and are all based on the same fundamental technology. Nearly all of these absorbent articles are comprised of a top sheet, a back sheet, and an absorbent core sandwiched between the top sheet and back sheet and located on the article at a position to receive bodily fluids. Optionally, a fluid acquisition layer may also be disposed between the top sheet and the absorbent core. The acquisition layer, for example, acquisition fibers, usually is incorporated in the absorbent articles to provide better distribution of liquid, increased rate of liquid absorption, reduced gel blocking, and improved surface dryness. A wide variety of acquisition fibers are known in the art. Included among these are synthetic fibers, a composite of cellulosic fibers and synthetic fibers, and cross-linked cellulosic fibers. Cross-linked cellulosic fiber is preferred because it is abundant, it is biodegradable, and it is relatively inexpensive.

Absorbent core materials are characterized in exhibiting favorably fluid absorbency and retention properties. These super absorbent materials are usually in the form of granules, beads, fibers, bits of film, globules, etc. Such super absorbent materials generally are polymeric gelling materials that are capable of absorbing and retaining large quantities of liquid relative to their own weight, even under moderate pressure. The super absorbent material generally is a water-insoluble but water absorbing polymeric substance capable of absorbing water in an amount which is at least ten times the weight of the substance in its dry form. In one type of super absorbent material, the particles or fibers may be described chemically as having a back bone of natural or synthetic polymers which hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated, or phosphorylated, causing the super absorbent polymers (SAPs) to be highly hydrophilic. Such modified polymers may also be cross-linked to reduce their water solubility.

The ability of a super absorbent material to absorb liquid typically is dependent upon the form, position, and/or manner in which particles of the super absorbent material are incorporated into the absorbent core. Whenever the super absorbent core is wetted, it swells and forms a gel. Gel formation can block or reduce liquid transmission into the interior of the absorbent core, a phenomenon called “gel blocking.” Gel blocking prevents liquid from rapidly diffusing or wicking past the blocking particles of SAP (e.g., those particles that have swelled and touched an adjacent swelled particle), causing unutilized portions of a partially hydrated core to become inaccessible to multiple doses of fluid. Further absorption of liquid by the absorbent core must then take place via diffusion process. This is typically much slower than the rate at which fluid enters the core, such as during urination. Gel blocking often leads to leakage from the absorbent article well before all of the absorbent material in the core is fully saturated.

In order to prevent underutilization of absorbent materials various solutions have been proposed which increase or maximize utilization of absorbent materials. In one such solution, a non-gelling fluid management layer is provided below or above the absorbent core to assist in distributing fluid over the entire core surface. However, even if full utilization of the absorbent core is achieved, fluid leakage out of the absorbent core remains a problem. Leakage occurs when the fluid escapes the absorbent core either through saturation, wicking, or compression due to dynamic motion of the wearer. When this happens, fluid from the core reaches the skin of the wearer and can even leak out of the absorbent article on to the wearer's clothes.

A great deal of effort and expense has been dedicated to address the problem of core fluid leakage. As a result various absorbent article designs have been proposed and patented that attempt to reduce and/or eliminate the problem of leakage. In one such design, extendible leg cuff barriers are used to provide fluid barrier to prevent fluid and solid waist products from leaking out of the absorbent article. However, such a design does not reduce leakage from the core itself, rather it prevents leakage out of the absorbent article. Moreover, this solution is most effective at preventing solid waist from escaping the absorbent article. Other leakage preventing core designs are disclosed in, for example, U.S. Pat. No. 6,786,895 (fluid barrier), U.S. Pat. No. 6,677,498 (wicking barrier and central rising member), U.S. Pat. No. 6,624,340 (longitudinally extending leakage preventing walls on two sides, U.S. Pat. No. 6,613,955 (wicking barrier leg cuffs), U.S. Pat. No. 6,592,563 (leakage preventing sidewalls on latitudinal sides of the fluid receiving side) and U.S. Pat. No. 6,548,732 (hydrophobic leak protection zones).

Other similar article designs include fluid impervious flaps arranged longitudinally on either side of the absorbent core to prevent egress of fluid beyond the core. However, all of these designs require additional materials, complicate the manufacturing process and increase the incremental cost of each absorbent article. In still further designs, fluid impervious channels are placed in the absorbent core creating a system of barriers which compartmentalizes portions of the absorbed fluid. However, like the flaps, this requires a more complex manufacturing process and increases the incremental article cost.

The description herein of various advantages and disadvantages associated with known apparatus, methods, and materials is not intended to limit the scope of the invention to their exclusion. Indeed, various embodiments of the invention may include one or more of the known apparatus, methods, and materials without suffering from their disadvantages.

### SUMMARY OF THE INVENTION

Therefore, it would be desirable to provide an absorbent article having an absorbent core which reduces and preferably eliminates side leakage while incorporating a relatively simple and economical design. It would also be desirable to provide an absorbent core for use with various absorbent articles which provides protection against lateral leakage out of the core. An additional desirable feature would be to provide an absorbent core for use with absorbent articles, which reduces and preferably minimizes leakage during compression and/or movement of the absorbent core when it contains fluid.

It is therefore a feature of an embodiment of this invention to provide an absorbent article having an improved ability to retain fluid and prevent lateral leakage out of the absorbent core. It is an additional feature of an embodiment of this invention to provide an absorbent article having an absorbent core with a fluid barrier surrounding at least the bottom and sides thereof. An additional feature of this invention is to provide an absorbent article having an absorbent core that is relatively simple and inexpensive to manufacture relative to other absorbent articles with leakage preventing designs.

In one embodiment according to this invention, an absorbent article is provided. The absorbent article according to this embodiment comprises a back sheet layer, a top sheet layer, an absorbent core supported between at least a portion of the back sheet layer and the top sheet layer, a C-shaped fluid impervious layer that surrounds at least the back sheet facing surface of the absorbent core and side surfaces of the core, and a fluid pervious layer that covers at least the remaining surfaces of the core.

In another embodiment according to this invention, an absorbent core for use with an absorbent article is provided. The absorbent core according to this embodiment, comprises a volume of an absorbent material defined by a top body facing surface, a back outer facing surface and side surfaces, wherein at least a portion of the top body facing surface comprises a fluid pervious layer and the remaining surfaces comprise a fluid impervious layer.

In yet another embodiment according to this invention, an method of manufacturing an absorbent article is provided. The method according to this embodiment comprises providing a top sheet layer, providing a back sheet layer, providing an absorbent core between at least portions of the top sheet layer and the back sheet layer as an integral absorbent article, providing a fluid permeable layer on at least a portion of one surface of the core, and providing a fluid impermeable layer on the remaining surfaces of the core, thereby encasing the core in a combination of fluid permeable and fluid impermeable materials.

In still a further embodiment according to this invention, an absorbent article having a C-folded absorbent core is provided. The absorbent article according to this embodiment comprises a back sheet layer, a top sheet layer, an absorbent core disposed between at least portions of the top sheet layer and the back sheet layer, a fluid pervious layer covering at least a portion of the surface of the absorbent core facing the top sheet layer, and a fluid impervious layer covering the remaining surfaces of the absorbent core, wherein the fluid impervious layer comprises a C-like cross sectional shape surrounding the absorbent core.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a perspective schematic view of a conventional absorbent article having an absorbent core in accordance with various exemplary embodiments of this invention;

FIG. 2 is a cross sectional view of an absorbent pad according to at least one exemplary embodiment of this invention;

FIG. 3 is a cross sectional view of an absorbent pad according to at least one other exemplary embodiment of this invention

FIG. 4 is a cross sectional view of an absorbent pad according to at least one additional exemplary embodiment of this invention

FIG. 5 is a cross sectional view of an absorbent pad according to at least a further exemplary embodiment of this invention;

FIG. 6 is a cross sectional view of an absorbent pad according to yet another exemplary embodiment of this invention;

FIG. 7 is a cross sectional view of an absorbent pad according to additional exemplary embodiments of this invention;

FIG. 8 is a cross sectional view of an absorbent pad according to even further exemplary embodiments of this invention; and

FIG. 9 is a plan view of an apparatus for use in forming an absorbent articles having a leakage resisting core in accordance with exemplary embodiments of this invention.

### DETAILED DESCRIPTION OF THE INVENTION

The following description is intended to convey a thorough understanding of the invention by providing specific embodiments and details involving absorbent articles and methods of manufacturing absorbent articles. It is understood, however, that the invention is not limited to these specific embodiments and details, which are exemplary only. It further is understood that one possessing ordinary skill in the art, in light of known systems and methods, would appreciate the use of the invention for its intended purposes and benefits in any number of alternative embodiments, depending upon specific design and other needs.

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment.

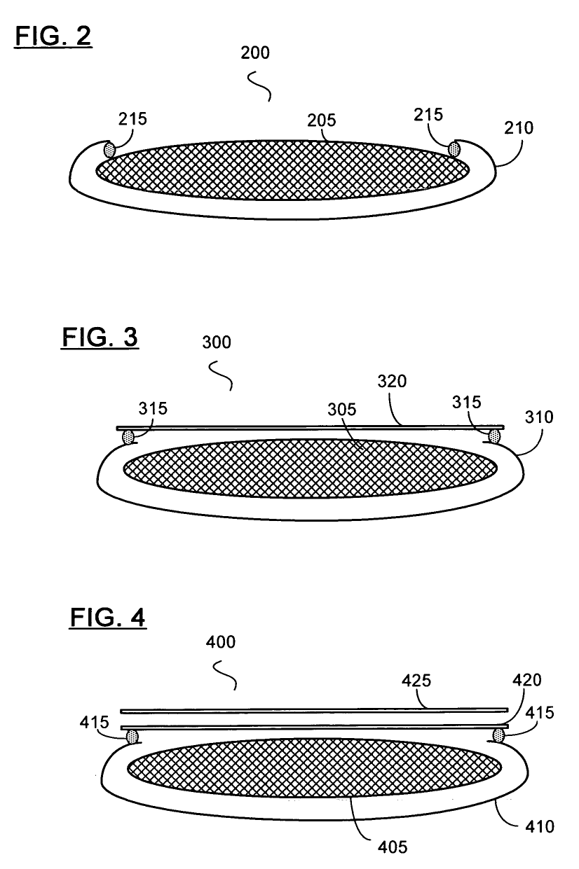

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention. In addition, upper and lower layer refers to the ultimate configuration of the absorbent core, a preferred cross-sections of which are illustrated in FIGS. 2-8.

The term “component” can refer, but is not limited, to designated selected regions, such as edges, corners, sides or the like; structural members, such as elastic strips, absorbent pads, stretchable layers or panels, layers of material, or the like; or a graphic.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any substantially continuous fiber. Tow fibers typically are used in the manufacture of staple fibers, and preferably are comprised of natural and/or synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow. Tow as used in the context of the present invention also encompasses modified tow fibers that have been either surface or internally modified (chemically or otherwise) to improve various desired properties of the fibers (e.g., wicking, etc.).

Throughout this description, the expression “super absorbent polymer” (“SAP”) or “super absorbent material” refers to any polymeric material that is capable of absorbing large quantities of fluid by forming a hydrated gel. Super absorbent polymers are well-known to those skilled in the art as substantially water-insoluble, absorbent polymeric compositions that are capable of absorbing large amounts of fluid (e.g., 0.9% solution of NaCl in water, or blood) in relation to their weight and forming a hydrogel upon such absorption. Super absorbent polymers also can retain significant amounts of water under moderate pressures. Super absorbent polymers generally fall into three classes, namely, starch graft copolymers, cross-linked carboxymethylcellulose derivatives, and modified hydrophilic polyacrylates. Examples of such absorbent polymers are hydrolyzed starch-acrylonitrile graft copolymer; a neutralized starch-acrylic acid graft copolymer, a saponified acrylic acid ester-vinyl acetate copolymer, a hydrolyzed acrylonitrile copolymer or acrylamide copolymer, a modified cross-linked polyvinyl alcohol, a neutralized self-cross-linking polyacrylic acid, a cross-linked polyacrylate salt, carboxylated cellulose, and a neutralized cross-linked isobutylene-maleic anhydride copolymer.

The present invention relates generally to absorbent articles, and in particular to absorbent articles having a top sheet, a back sheet, and an absorbent core disposed at least partially between the top sheet and the back sheet. The absorbent core of the invention preferably is comprised of a central fibrous layer and one or more upper and/or lower layers, whereby the central fibrous layer is a fibrous structure comprised of synthetic fibers and, optionally, SAP. The absorbent core is also surrounded in part by a fluid impervious layer. In a preferred embodiment, the fluid impervious layer surrounds the absorbent core on the back surface, side surfaces, and a portion of the top surface providing a substantially leak proof pocket around the absorbent core. Throughout this description and claims, the term fluid impervious layer, in the context of the absorbent core, may refer to any fluid impervious layer including a treated tissue layer, a woven or non-woven layer, or a fluid impervious film.

The invention also relates in general to a method of making an absorbent article that includes providing a top sheet material and a back sheet material. The method also includes preparing an absorbent core that contains at least one central fibrous layer comprised of a synthetic fibrous material and SAP. The absorbent core is surrounded in part with a fluid impervious layer. In a preferred embodiment, the absorbent core is further prepared by surrounding the bottom, sides and a portion of the top with a fluid impervious layer.

The present invention is premised in part on the discovery that surrounding at least a portion of the absorbent core, and preferably the bottom surface, side surfaces and a portion of the top surface, with a fluid impervious layer will significantly reduce the amount of lateral leakage from the absorbent core. Traditional absorbent cores that do not employ integral leakage protecting devices are ineffective at preventing core leakage. Modified core designs employing additional garment features such as flaps, fluid impervious leg elastics or other fluid containing schemes, fail to balance the goals of fluid retention and cost reduction by employing unnecessarily complicated and expensive designs. In contrast, in certain embodiments of the present invention, the absorbent core is manufactured with a fluid impervious outer sheet on at least the bottom and sides and fluid pervious top layer. Therefore, once the absorbent core becomes wetted or saturated, the fluid impervious materials maintain the fluid within the core. This allows the product designer to prevent leakage out of the core into other areas of the garment with a near minimal impact on product complexity and manufacturing costs.

The invention now will be described with reference to the attached drawings illustrating preferred embodiments of the invention. For clarity, features that appear in more than one Figure have the same reference number in each Figure. For sake of simplicity and ease of explanation, the invention will be discussed in the context of diaper-type absorbent articles. However, it should be appreciated by those possessing ordinary skill in the art, that the principles disclosed herein may be applied equally to any other type of absorbent article giving the term its broadest reasonable interpretation as defined herein.

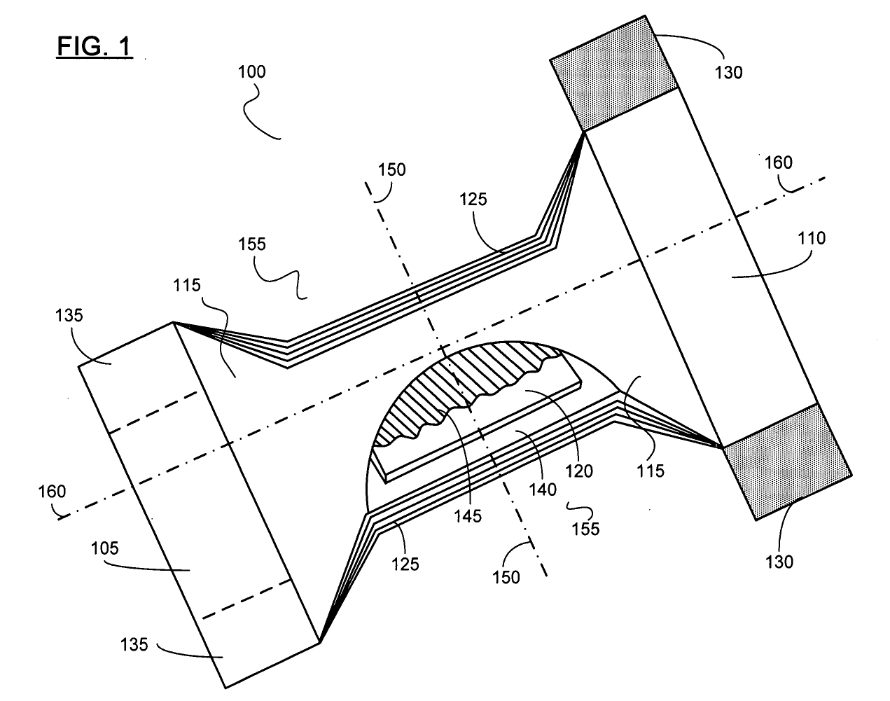

Referring now to FIG. 1, a typical absorbent article 100 is illustrated in a partial cut away perspective view. The absorbent article depicted in FIG. 1 is an infant diaper, however, as discussed above, this depiction is not intended to limit the invention. Those skilled in the art will appreciate that the invention will cover other types of absorbent articles as well. The absorbent article 100 of FIG. 1 is depicted in a generally flattened position, with the body-facing side facing up and the outer side facing down, and with the various elastic components in depicted in the relaxed position with the effects of elasticity removed (typically relaxed elastics would cause the garment to bunch or contract around the locations of the elastics). In the flattened position, the absorbent article 100 has a generally hourglass configuration. Diapers in particular will typically have either an hourglass, rectangular, or even T shape.

As used in this disclosure, the longitudinal axis, axis 160 in FIG. 1, of an absorbent article and/or absorbent core is the dimension corresponding to the front to rear axis of the wearer, and the transverse axis, 150 in FIG. 1, is the side-to-side axis of the wearer. The side to side axis 150 will generally provide a cross sectional view of the absorbent article and/or absorbent core.

The absorbent article 100 of FIG. 1 is constructed essentially of a top sheet 115, a back sheet 140 and an absorbent core 120 secured between at least portions of the top sheet 115 and back sheet 140. The top sheet layer 115 typically consists of a hydrophobic, yet fluid pervious web material that allows fluid to pass through to the absorbent core without absorption and that is relatively comfortable against the skin of the wearer. The back sheet layer 140 is typically comprised of a fluid impervious layer such as a poly-based web material which is typically coated with a thin external cloth-like layer designed to simulate a fabric material and de-emphasize the garment's plasticity. The top sheet layer 115 and the back sheet layer 140 may be constructed from a wide variety of materials known in the art. In the invention is not intended to be limited to any specific materials for these components. The top sheet layer 115 and back sheet layer 140 can be shaped and sized according to the requirements of each of the various types of absorbent garments, or to accommodate various user sizes. In an embodiment of the invention in which the absorbent article is a diaper or adult incontinence brief, the combination of the top sheet 115 and the back sheet 140 may have an hourglass shape, as seen in FIG. 1, or a rectangular, trapezoidal, “T” shape, or any other shape.

Due to the wide variety of backing and liner sheet construction and materials currently available, the invention is not intended to be limited to any specific materials or constructions of these components. The back sheet 140 preferably is made from any suitable pliable liquid-impervious material known in the art. Typical back sheet materials include films of polyethylene, polypropylene, polyester, nylon, and polyvinal chloride and blends of these materials. For example, the back sheet can be made of a polyethylene film having a thickness in the range of 0.02-0.04 mm. The back sheet 140 may be pigmented with, for example, titanium dioxide, to provide the garment 100 with a pleasing color or to render the back sheet 140 opaque enough that exudates being contained by the garment 100 are not visible from outside the garment. In addition, the back sheet 140 may be formed in such a manner that it is opaque, for example, by using various inert components in the polymeric film and then biaxially stretching the film. Other back sheet materials will be readily apparent to those skilled in the art. The back sheet 140 preferably has sufficient liquid imperviousness to prevent any leakage of fluids. The required level of liquid imperviousness may vary between different locations on the garment 100.

The back sheet 140 may further comprises separate regions having different properties. In a preferred embodiment, portions of the back sheet 140 are air permeable to improve the breathability, and therefore comfort of the garment 100. The different regions may be formed by making the back sheet 140 of a composite of different sheet materials, chemical treatment, heat treatment, or other processes or methods known in the art. Some regions of the back sheet 140 may be fluid pervious. In various exemplary embodiments of the invention, the back sheet 140 is fluid impervious in the crotch region 155 but is fluid pervious in the waist regions 105 and 110. The back sheet 140 may also be made from a laminate of overlaid sheet materials.

The moisture pervious top sheet 115 can be comprised of any suitable relatively liquid-pervious material known in the art that permits passage of liquid there through. Non-woven liner sheet materials are exemplary because such materials readily allow the passage of liquids to the underlying absorbent core 120. Examples of suitable liner sheet materials includes non-woven spun bond or carded webs of polypropylene, polyethylene, nylon, polyester and blends of these materials.

The back sheet 140 may be covered with a fibrous, non-woven fabric such as is disclosed, for example, in U.S. Pat. No. 4,646,362 issued to Heran et al., the disclosure of which is incorporated by reference in its entirety and in a manner consistent with this disclosure. Materials for such a fibrous outer liner include a spun-bonded non-woven web of synthetic fibers such as polypropylene, polyethylene or polyester fibers, a non-woven web of cellulosic and textile fibers, a spun-bonded non-woven web of synthetic fibers such as polypropylene, polyethylene or polyester fibers mixed with cellulosic, pulp fibers, or textile fibers, or melt blown thermoplastic fibers, such as macro fibers or micro fibers or polypropylene, polyethylene, polyester or other thermoplastic materials or mixtures of such thermoplastic macro fibers or micro fibers with cellulosic, pulp or textile fibers. Alternatively, the back sheet 140 may comprise three panels wherein a central poly back sheet panel is positioned closest to the absorbent core 120 while outboard non-woven breathable side back sheet panels are attached to the side edges of the central poly back sheet panel. Alternatively, the back sheet 140 may be formed from microporous poly cover stock for added breathability.

The layout of the absorbent article 100 is such that there is a front waist region 105, a rear waist region 110 and a tapered middle portion 155 connecting the front waist region 105 and rear waist region 110. The labels front and rear are derived from their position on the wearer's body during use. The tapered crotch region 155 contains one or more leg elastics 125 which run the length of either edge of the tapered crotch region 155. The leg elastics 125 provide a secure fit on the wearer and assist in trapping liquid and solid waist released by the wearer during use. The tapered crotch region 155 is designed to receive and retain fluid though a fluid absorption and retention system. The fluid absorption and retention system consists of an absorbent core 120 located under the top sheet layer 115. Optionally, a fluid acquisition layer 145 may be located on a top surface of the absorbent core 120 to assist in absorption of fluid to the core. Typically, such a fluid acquisition layer 145 will have a higher absorption rate than the material used in the core 120 to assist in pulling fluid away from the wearer. Underneath the absorbent core is the back sheet layer 140 which, as noted above, is typically constructed of a fluid impervious material. In order to expedite the manufacturing process, the top sheet layer 114, the absorbent core 120, the back sheet layer 140 and any acquisition layer 145 are joined into a single garment by joining multiple webs of stock materials which are held together with adhesives. Through such a process, several hundred units of absorbent garments may be produced in a single minute.

The absorbent core 120, including the optional fluid acquisition layer 145, can comprise one or more layers. In addition, one or more additional layers (not shown) may be disposed either under or over the absorbent core 120, such as between the absorbent core 120 and the back sheet layer 140 or between the absorbent core 120 and the top sheet layer 115. The additional layers may include a fluid transfer layer, a fluid handling layer, a storage layer, a wicking layer, a fluid distribution layer, and any other layer(s) known to those having ordinary skill in the art.

Although the absorbent core 120 depicted in FIG. 1 has a substantially rectangular shape, other shapes may be used such as an hour glass shape, or elliptical shape. The shape of the absorbent core 120 may be selected to maximize absorbency with a minimum amount of material. The absorbent core 120 may be affixed to the top sheet 115, the back sheet 140, or any other suitable part of the garment 100 by any method known in the art, in order to fix the absorbent core 120 in place. In addition to any individual layers within the absorbent core 120, the overall core 120 may be enclosed within a tissue wrapping, as disclosed in U.S. Pat. No. 6,068,620, the disclosure of which is hereby incorporated by reference in its entirety.

Though the absorbent core 120 depicted in FIG. 1 is shown as terminating prior to the either the front waist region 105 or the rear waist region 110, the absorbent core 120 may also extend into either of these portions. A typical absorbent core 120 will usually contain at least a central fibrous layer, and one or more other layers, such as, for example, a fibrous layer, a fluid management layer and a tissue layer. The central fibrous layer of the absorbent core 120 will comprise a fibrous structure. Central fibrous layers of this type generally are known in the art, and exemplary absorbent cores are described in U.S. Pat. Nos. 6,068,620 and 5,281,207, both issued to Chmielewski, and U.S. Pat. No. 5,863,288, issued to Baker, the disclosures of each of which are hereby incorporated by reference in their entirety in a manner consistent with this disclosure.

Certain fibrous and particulate additives preferably are used as constituent elements of the central fibrous layer. Fibrous additives of the central fibrous layer preferably include, but are not limited to, synthetic fibers, such as cellulose ester fibers, cellulose acetate fibers, rayon fibers, lyocell fibers, polyacrylonitrile fibers, polyolefin fibers, surface-modified (hydrophilic) polyester fibers, surface-modified poyolefin/polyester bicomponent fibers, surface-modified polyesters/polyester bicomponent fibers, or natural fibers, such as cotton or cotton linters, or combinations or blends thereof. The fibrous additives are preferably synthetic fibers. Of the foregoing, cellulose acetate is the most preferred synthetic fibrous additive for use in the central fibrous layer because it is biodegradable and relatively inexpensive. In addition, rayon, lyocell, and polyacrylonitrile have similar properties to cellulose acetate and are alternatively preferred. The remaining synthetic fibers, polyolefin fibers, surface-modified polyolefin/polyester bicomponent fibers, and surface-modified polyester/polyester bicomponent fibers are also believed to be effective fibrous additives.

Synthetic fibrous components of the absorbent core 120 are preferably comprised of tow fiber, and most preferably a crimped tow of cellulose acetate, polypropylene, polyester, or mixtures thereof. Before making the absorbent core that includes a tow fiber, the tow fiber typically is unwound and opened, and then fed to the core forming station to provide a fibrous mass of material. Persons of ordinary skill in the art are aware of techniques available to open tow fibers and form the opened fibers into a fibrous mass. In addition, the fibrous component of the central fibrous layer may include a low-density roll good made in a separate process. Still further yet, the fibrous component could also include a carded web formed on-line. Optionally, it is advantageous to introduce from about 1-5% of a thermally bondable fiber into the fibrous component of the central fibrous layer for wet strength and core stability in use. In addition to the tow material used as the fibrous component in the central fibrous layer, other fibrous components also may be used.

In accordance with the present invention, the absorbent core preferably comprises a tow fiber, and preferably, a substantially continuous crimped filament tow. This fiber structure has high structural integrity, and as such, is distinct from a matrix of discontinuous fibers described as fluff, or fluff pulp in the prior art. The high structural integrity enables the production of stronger webs than those formed from discontinuous fibers, which in turn are believed to enable the production of thinner absorbent pads. In addition, the use of such fibers enables the production of ultra low density absorbent cores, when compared to absorbent cores prepared by dispersing SAP particles in fluff.

The synthetic fiber can be any substantially continuous or discontinuous thermoplastic filament fiber that is capable of being used in combination with SAP in an absorbent core. Preferably, polypropylene or cellulose ester fiber is used as the fibrous material in central fibrous layer. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include some ability to absorb moisture, (but absorptive capacity is not necessarily required), preferably is biodegradable, and is influenced not only by the substitute groups but also by the degree of substitution. The relationship between substitute groups, degree of substitution and biodegradability is discussed in W. G. Glasser et al., BIOTECHNOLOGY PROGRESS, vol. 10, pp. 214-219 (1994), the disclosure of which is incorporated herein by reference in its entirety.

The synthetic fiber useful in the present invention is beneficially moisture-absorbent and biodegradable. Accordingly, cellulose acetate tow is typically preferred for use in the invention. Typically, the denier per fiber (dpf) of the fiber will be in the range of about 1 to 25, preferably about 3 to 15, and most preferably about 6 to 7. For the same weight product, filaments of lower dpf may provide increased surface area and increased moisture absorption. Total denier may vary within the range of about 5,000 to 60,000, more preferably from about 20,000 to about 40,000, and most preferably from about 20,000 to about 30,000, depending upon the process used.

It is particularly preferred in the invention to use tow having crimped filaments. Tow materials having crimped filaments are typically easier to open. Separation of filaments resulting from bloom advantageously results in increased available filament surface area for superabsorbent material immobilization and increased moisture absorption. Gel blocking also may be reduced by using crimped tow in the central fibrous layer. As therefore may be understood, more crimp is typically better, with in excess of about 20 crimps per inch being usually preferred. Substantially continuous filament, cellulose ester tow having crimped filaments with about 25 to 40 crimps per inch, is commercially available from Celanese Acetate, Charlotte, N.C.

It is preferred in the present invention that the tow fibers in central fibrous layer have an average length generally about the same length as the absorbent core. Typically, the tow is a substantially continuous filament that is cut to length during manufacture of the core. The average diameter of the tow fibers typically is expressed as the cross sectional area of the fibers, although the width of the fibers preferably is within the range of from about 50 to about 200 mm, more preferably from about 75 to about 150 mm, and most preferably from about 85 to about 120 mm. The cross sectional area is based on the denier and density of the fibers. For example, the denier per foot (dpf) and density (typically an acetate polymer density is about 1.32 g/cm³), can be used to calculate the cross sectional area. A 3.0 dpf acetate polymer fiber has a cross sectional area 2.525×10⁻⁶ cm².

The central fibrous layer may optionally comprise discontinuous synthetic fibers. As used herein, the term “discontinuous” fibers means fibers that have an average length less than the length of the absorbent core. As such, the central fibrous layer 284 may comprise, for example, a non-woven mat or web of discontinuous synthetic fibers. The fibers may be provided to the absorbent core 120 as a substantially continuous tow fiber, and then cut to length and formed into a web during the processing of the absorbent core 120, or the fibrous web may be formed off-line, and provided to the absorbent core 120 as a roll-good material.

The central fibrous layer may optionally contain super absorbent polymer (SAP). Any super absorbent polymer now known or later discovered may be used in central fibrous layer so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAP are water swellable polymers of water soluble acrylic or vinyl monomers cross linked with a polyfunctional reactant. Also included are starch modified polyacrylic acids and hydrolyzed polyacrylonitrile and their alkali metal salts. A more detailed recitation of super absorbent polymers is found in U.S. Pat. No. 4,990,541 to Nielsen, the disclosure of which is incorporated herein by reference in its entirety.

Commercially available SAPs include a starch modified super absorbent polymer available under the trade name HYSORB® from BASF Aktiengesellschaft, Ludwigshafen, Germany. Other commercially available SAPs include a super absorbent derived from polypropenoic acid, available under the trade name DRYTECH® 520 SUPER ABSORBENT POLYMER from The Dow Chemical Company, Midland Mich.; AQUA KEEP, and AQUA KEEP SA60S, manufactured by Sumitomo Seika Chemicals Co., Ltd., Osaka Japan.; ARASORB manufactured by Arakawa Chemical (U.S.A.) Inc.; FAVOR manufactured by Stockhausen Inc.; DIAWET, commercially available from Mitsubishi Chemicals, Japan; FLOSORB, available from SNF Floerger, France, AQUALIC, available from Nippon Shokubai, Osaka, Japan.

The SAP may be provided in any particle size, and suitable particle sizes vary greatly depending on the ultimate properties desired. It has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in its entirety

It is preferred in a SAP-containing central fibrous layer to use relatively coarse fibers having a low basis weight such that the pore size of the matrix formed by the mass of tow fibers does not entrain some or most of the SAP, but rather allows the SAP to fall freely through the matrix. The basis weight of preferred fibers used in the present invention ranges from about 20 to about 200 g/m², more preferably from about 50 to about 100 g/m², and most preferably from about 70 to about 80 g/m².

To maintain high SAP concentrations, the concentration of fibrous material in the central layer of the absorbent core 120 of the invention preferably is about 5%-99%, more preferably about 80%-99%, and most preferably about 90-99%. Most preferably, the central fibrous layer comprises from about 0%-50% SAP and from about 50%-99% fibrous materials selected from the foregoing group, or the fibrous components discussed below.

Particulate additives may be added to central fibrous layer in addition to or as a substitute for the foregoing fibrous additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity/hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Pat. No. 6,068,620.

If desired, an absorptive pad of multiple layer thickness, may be provided. To this end, the tow may be, for example, lapped or crosslapped in accordance with conventional procedures. In this way, a superabsorbent, absorptive material of a desired weight and/or thickness may be provided. The specific weight or thickness will depend upon factors including the particular end use. It is especially preferred that the crimped cellulose acetate tow material be opened and then mixed with the SAP particles to form the central fibrous layer.

The total basis weight of the absorbent core 120 including fibrous materials, SAP, extrudate layer, base layers, additional layers, and additives, can be anywhere from about 50-1,000 grams per square meter. The most preferred total basis weights of the absorbent core 120 are about 300-600 grams per square meter.

In addition to the other configurations, additional layers may be present in the absorbent core 120. For example, absorbent core 120 may include an additional layer disposed above, below or between any of layers of the absorbent core 120, such as above the central fibrous layer, and/or below central fibrous layer. Any additional layer can be used, including any layer selected from a fluid acquisition layer, a distribution layer, an additional fibrous layer optionally containing SAP, a wicking layer, a storage layer, or combinations and fragments of these layers. Such layers may be provided to assist with transferring fluids to the absorbent core 120, handling fluid surges, preventing rewet, containing absorbent material, improving core stability, or for other purposes. Persons of ordinary skill in the art are familiar with the various additional layers that may be included in an absorbent article, and the present invention is not intended to be limited to any particular types of materials used for those layers. Rather, the invention encompasses all types of wicking layers, all types of distribution layers, etc., to the extent that type of layer is utilized.

The absorbent core 120 may contain upper and lower layers, which encase the central fibrous layer, a sub-layer, and SAP. These layers may be made of, for example, tissue, film or nonwoven, but may also form the top sheet and back sheet of the absorbent garment, or any other layers. The upper and lower layers preferably are wider than the central fibrous layer that forms the absorbent core, and their side portions preferably are sealed to one another by bonding, by crimping or by both to prevent release of opened tow and particles of SAP. The upper and lower layers preferably are comprised of the same material folded over onto itself, and only the open end sealed by crimping or bonding. The absorbent core 120, comprising the assembly of the central fibrous layer and a sub-layer including the opened tow and SAP, may be further processed as it is conveyed through the assembly line for inclusion into absorbent garments. For example, the absorbent core 120 may be severed into individual absorbent cores, and the severed ends may be crimped or bonded or both to prevent the SAP from exiting the ends.

Crimping, bonding or both can be performed on the absorbent core 120 of the invention using conventional means. For example, the lateral side edges, and longitudinal edges can be sealed together by intermittent or substantially continuous application of adhesive to the respective portions of the upper and lower layers using any device capable of applying adhesives to a continuous moving web of material. The lateral and/or longitudinal edges then can be pressed together to form a seal. The seal also can be formed ultrasonically, or the respective edges (lateral and/or longitudinal) can be crimped using crimping rollers or any other crimping device known to those having ordinary skill in the art. Using the guidelines provided herein, those skilled in the art will be capable of sealing the lateral and/or longitudinal edges of absorbent core 120 using bonding, crimping, or both.

It is possible in the present invention to mechanically and/or thermally work the absorbent core 120 to make it more flexible. Any technique presently known in the art or later discovered may be used to work the absorbent core. For instance, the absorbent core 120 may be embossed or texturized using a continuous or intermittent calendaring apparatus. Other useful techniques include, for example, compression, thermal bonding, and ultrasonic bonding. Optionally, the top sheet 115 and/or the back sheet 140 may be worked with the absorbent core 120.

It is also possible in the present invention that the absorbent core 120 be folded as it is disposed in the absorbent garment. The absorbent core 120 can be folded in any suitable manner, including any and all of those disclosed in U.S. Pat. No. 6,068,620. Suitable folds include “C” folds, “G” folds, “U” folds, “A” folds, pleats or “W” folds, and the like.

With continued reference to FIG. 1, on the rear waist region 110, there are a pair of fastening tabs 130 which, when the garment is attached to the wearer, mate with the front waist region portion 105, on mating portions 135 respectively. In various exemplary embodiments, mating is facilitated to through the use of a mechanical fastener such as VELCRO, and, in various other embodiments, the fastening tabs have an adhesive disposed thereon to facilitate fastening. Any fastening system know in the art will be acceptable. Moreover, the fastening system may include a reinforcement patch below the front waist portion 105, near the mating portions 135 so that the diaper may be unfastened and checked for soiling without compromising the ability to reuse the fastener. Alternatively, other absorbent article fastening systems are also possibly, including safety pins, buttons, and snaps. Furthermore, elastics may also be utilized in either the fastening tabs 130, the rear waist region 110, the front waist region 105 or mixtures thereof to enhance comfort and fitment.

FIG. 2 is a cross sectional view taken along transverse line 150-150 in FIG. 1 of a leakage reducing absorbent core 200 in accordance with at least one exemplary embodiment of this invention. The absorbent core 200 illustrated in FIG. 2 includes an absorbent core material portion 205 and a fluid barrier portion 210 which surrounds the core portion 205 in a C-like pattern. In various exemplary embodiments, the fluid barrier portion 210 will serve as the external wrapper for a portion of the absorbent core 200. However, in various other exemplary embodiments, the absorbent core portion 205 will be a unitized core with a tissue wrapper surrounding the absorbent materials contained within the core portion 205, and the fluid barrier portion 210 will simply be an additional layer surrounding a part of the core portion 205.

Optionally, and as shown in FIG. 2, an adhesive bond 215 may be deposited on the core to secure the ends of the fluid barrier portion 210 at the appropriate location on the top surface of the absorbent core portion 205. It should be noted that though an adhesive bond 215 is shown in FIG. 2, that any other adhesive, mechanical, thermal or combination fastening means such as adhesives, pressing, thermal bonding, crimping, sonic bonding may be utilized with embodiments of this invention. Generally, though not necessary, due to the incongruent nature of the absorbent materials used in the core portion 205, the absorbent core portion 205 will not comprise a perfectly geometric shape. Therefore, as illustrated in the absorbent core 200 of FIG. 2, the core portion 205 is generally elliptical in shape. The fluid barrier portion 210 surrounds the core portion 205 such that the fluid barrier portion subtends an angle ranging from zero (horizontal) up through in excess of Π/2 radians (>90 degrees) on either side of the core portion 205. Therefore, the fluid barrier portion 210 not only covers the bottom and sides of the core portion 205, but also covers at least part of the top surface of the core portion 205. During the manufacturing and assembly, a portion of a roll of feedstock of absorbent core 200 is cut to provide sufficient length to run longitudinally from the front waist region to the rear waist region of the absorbent article covering the entire crotch area in which fluid is typically discharged. Though the absorbent core could also be protected by the fluid barrier portion on either end as well as on the sides, end coverage is less important because when worn, the open cut ends of the barrier face upwards. Therefore, fluid would typically only run or wick out of either end of the absorbent core when the wearer is at an inverted angle with respect to horizontal, or when extreme saturation has occurred.

FIG. 3 is a cross sectional view taken along transverse line 150-150 in FIG. 1 of a leakage reducing absorbent core 300 in accordance with at least one exemplary embodiment of this invention. The absorbent core 300 illustrated in FIG. 3 is similar to that of FIG. 2, in that it includes an absorbent core portion 305 and a C-shaped fluid barrier portion 310, however, it also includes it also includes a fluid acquisition layer 320 disposed over the core portion 320 and approximately co-terminal with either end of the fluid barrier portion 310. The fluid acquisition layer 320 may be a film or non-woven fluid acquisition layer. As in the absorbent core 200 of FIG. 2, adhesive bonds 315 may be utilized to secure the acquisition layer 320 and the fluid barrier portion 310 in their respective positions with respect to the core portion 305. By using a fluid acquisition layer 320, fluid is induced into the core portion 305 at a higher rate. Also, because the fluid acquisition layer is typically hydrophobic, it provides an air barrier between the fluid retaining core portion 305 and the body of the wearer.

FIG. 4 is a cross sectional view taken along transverse line 150-150 in FIG. 1 of a leakage reducing absorbent core 400 in accordance with at least one exemplary embodiment of this invention. The absorbent core 400 of FIG. 4 is similar to that of the absorbent core 300 shown in FIG. 3, in that it includes a core portion 405, a C-shaped fluid barrier portion 410 and a fluid acquisition layer 420. However, the absorbent core 400 also includes a second non-woven or apertured film laminated acquisition layer 425 which further assist in drawing fluid to the core portion 404 and away from the body of the wearer. As with previously illustrated embodiments, the absorbent core 400 of FIG. 4 utilizes adhesive bonds 414 to secure the terminal edges of the various layers to the core portion 405.

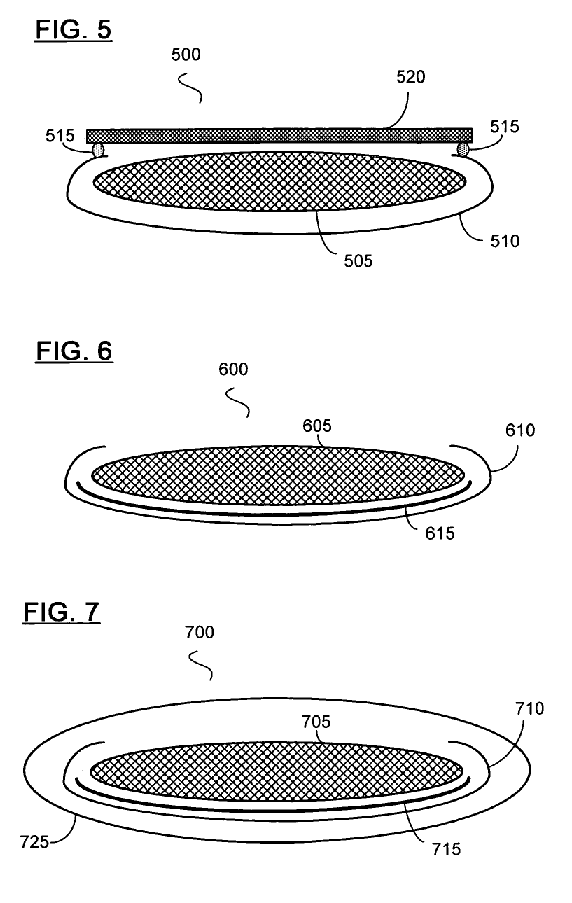

FIG. 5 is a cross sectional view taken along transverse line 150-150 in FIG. 1 of a leakage reducing absorbent core 500 in accordance with at least one exemplary embodiment of this invention. The absorbent core 500 of FIG. 5 is similar to that of previous embodiments in that includes a core portion 505 and a C-shaped fluid barrier portion 510, however, it also includes a single lane-apertured fluid impervious layer 520 that allows fluid to enter the core portion 505 through individual apertures. The lane-apertured material 520 is fused to the non-apertured solid fluid barrier material 510 by adhesive bond portions 515.

FIG. 6 is a cross sectional view taken along transverse line 150-150 in FIG. 1 of a leakage reducing absorbent core 600 in accordance with at least one exemplary embodiment of this invention. The absorbent core portion 600 of FIG. 6 is characterized in that it consists of a core portion 605, a C-shaped fluid barrier portion 610 and a fluid management layer 615. For purposes of example, the fluid management layer 615 is depicted as being located under the core portion 605 and partially covering the edges of the core portion 605. However, it should be appreciated that it may be desirable to cover more or less of the core portion 605 with the fluid management layer 615, including surrounding the entire core portion 605 with a sleeve-like fluid management layer 615. The fluid management layer 615 assists in dispersing fluid evenly throughout the core portion 605. By utilizing such a fluid management layer 615 under the core portion 605, effects from phenomena such as gel blocking may be reduced. Also, even in cores utilizing materials not subject to gel blocking, the use of a fluid management layer can helps to spread fluid away from the middle, where additional incoming fluids enter, to more remote portions of the core 605 helping to increase total core utilization and thereby increase fluid capacity and retention of the absorbent core 600.

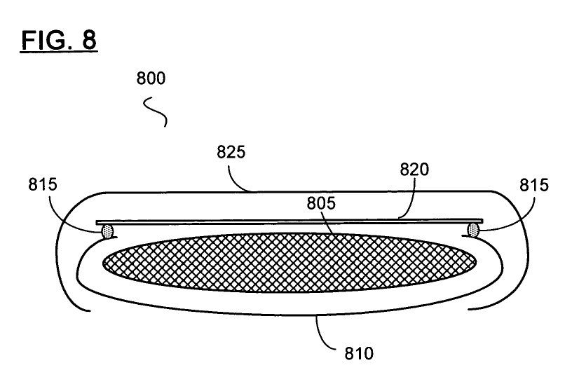

FIG. 7 is a cross sectional view taken along transverse line 150-150 in FIG. 1 of a leakage reducing absorbent core 700 in accordance with at least one exemplary embodiment of this invention. The absorbent core 700 shown in FIG. 7 includes a core portion 705, a C-shaped fluid barrier portion 710 and a non-woven or apertured film layer 715 which completely surrounds the core to create a unitized tube-like core structure. In various exemplary embodiments, the non-woven or apertured film layer 715 may be a rolled sheet which is folded around the core portion 705 and fluid barrier portion 710 and sealed with the use of an adhesive bond, sonic bond, thermal bond, crimping, pressing, or other thermal, mechanical or adhesive bonding means. Though not shown, the absorbent core illustrated in FIG. 7 may also include a fluid acquisition layer between the non-woven or apertured film 715 and the core portion 705 and/or a fluid management layer between the fluid barrier layer 710 and the core portion 705.

FIG. 8 is a cross sectional view taken along transverse line 150-150 in FIG. 1 of a leakage reducing absorbent core 800 in accordance with at least one exemplary embodiment of this invention. The absorbent core 800 of FIG. 8 includes a core portion 805, a C-folded fluid barrier portion 810, a non-woven or film fluid acquisition layer 820 joined with the fluid barrier layer 815 by adhesive bonds 815 and a C-folded non-woven or apertured film portion 825 covering at least the top of the absorbent core 800. Though not illustrated, the absorbent core 800 of FIG. 8 may also include a fluid management layer disposed between the fluid barrier layer 810 and the core portion 805.

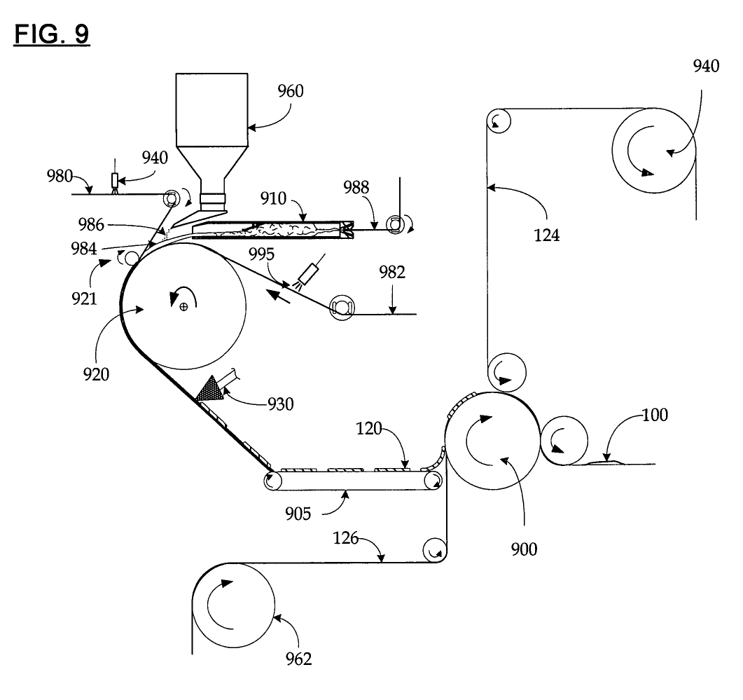

Referring now to FIG. 9, the invention also relates to a method of making an absorbent multi-layer core 120, and an absorbent article that includes providing a top sheet material 124 and a back sheet material 126. The method also includes preparing an absorbent multi-layer core 120 by disposing a central absorbent layer comprising a mixture of fibrous material and SAP between an upper layer 980 and a lower layer 982. The method includes disposing the absorbent multi-layer core 120 between the top sheet 124 and the back sheet 126.

FIG. 9 illustrates an apparatus useful in forming an absorbent article 100 in accordance with the present invention. Any type of tow fiber 988 can be supplied to the apparatus and, as conventional in the art, the tow fiber 988 typically is opened prior to forming a fibrous matrix. In this regard, the apparatus includes a tow opener and feeder 910 that is capable of opening any suitable tow material, expanding the tow fiber and feeding the tow fiber to the core forming station 920. Any suitable tow opener and feeder 910 can be used in the method of the invention.

The tow fibers 988 preferably are mixed with superabsorbent polymer (SAP) material 986 to form central absorbent layer 984. The SAP is fed to the core forming station 920 by any SAP feeder 960 capable of feeding the SAP 986 to the core forming station 920. Those skilled in the art are capable of designing a suitable SAP feeder 960 and nozzle configuration to provide adequate mixing of SAP material 986 and tow fibers 988 to form central absorbent layer 984.

The absorbent multi-layer core 120 can be formed at core forming station 920, where central absorbent layer 984, comprised of SAP material 986 and tow fibers 988, is disposed between an upper layer 980, and a lower layer 982. Upper and lower layers 980, 982 can be fed to core forming unit 920 using any supplying mechanism known in the art, and preferably are fed through one or more feed rollers. Adhesive can be applied to either upper layer 980 or lower layer 982, to both layers, or to neither layer, by an adhesive applicator 940. Again, any mechanism capable of supplying an adhesive, albeit a spray adhesive, or one that is “rubbed” on, can be used in the invention. Suitable adhesives 995 include any adhesive commonly employed in absorbent garments that is useful in adhering one or more tissue and/or non-woven materials together. It is particularly preferred to use construction adhesives, including HL-1258 by H. B. Fuller Company of St. Paul, Minn.; Findley 2031 and H2587-01 by Ato Findley Inc. of Wauwatosa, Wis.; and NS34-5665 by National Starch Co. of Bridgewater, N.J. Other adhesives that may be used in the invention include 34-578A, available from National Starch Co. of Bridgewater, N.J. Any of these adhesives may be used in all adhesive applications in the absorbent garment, or only in select applications as a construction adhesive for bonding parts of the garment as the top sheet, back sheet, absorbent core, and additional layer(s).

As the SAP material 986 and tow fibers 988 mix together to form central absorbent layer 984, which in turn is disposed between upper layer 980 and lower layer 982 at core forming station 920, some of these SAP particles may become affixed in the adhesive 995 when the absorbent multi-layer core 120 is passed through the one or more nip rollers 921 at the core forming station 920. The absorbent multi-layer cores 120 then are cut to length by cutting knife 930. Cutting knife 930 can be any suitable cutting device capable of cutting absorbent multi-layer core 120 of the invention. For example, cutting knife 930 can be comprised of a set of rollers; one being an anvil, and another having a knife attached at one point on the roller, whereby the diameter of the roller is selected to coordinate with the speed at which absorbent multi-layer cores 120 are formed. The knife roller and anvil roller then can rotate at the same speed as the line speed to cut the absorbent multi-layer core 120 at select areas to form uniform length cores 120. Skilled artisans are capable of designing a suitable cutting knife 930 given the specifics of each article forming assembly line.

The absorbent multi-layer cores 120 then are transported to forming station 900 via core conveyor 905. Top sheet material 124 may be supplied to forming station 900 by top sheet supply mechanism 940, which can be any supply mechanism capable of supplying top sheet 124 to forming station 900. Preferably, top sheet material 124 is supplied via a supply roller 940 and select feed or guide rollers. Back sheet material 126 likewise can be supplied to forming station 900 by back sheet supply mechanism 960, which can be any supply mechanism capable of supplying back sheet 126 to forming station 900. Preferably, back sheet material 126 is supplied via a supply roller 962 and select feed or guide rollers. Forming station brings together the respective components of absorbent article 100 by disposing absorbent multi-layer core 120 between top sheet material 124, and back sheet material 126. The final absorbent article 100 then may be cut and folded to the appropriate size and shape downstream from forming station 900.

### EXAMPLE

Table 1.1 below summarizes the results of an experiment conducted by the inventors of this application in which a traditional absorbent core, that is one not employing the leakage prevention technique of the present invention, was compared side-by-side with a novel absorbent core having a C-shaped fluid barrier surrounding the outer periphery of the core on the bottom, sides and a portion of the top, such as the absorbent core illustrated in FIG. 2. The experiment was conducted by utilizing two otherwise equivalent 10″ long sections of absorbent core, that is one manufactured under conventional techniques and one manufactured with a C-shaped fluid barrier in accordance with embodiments of this invention. In each of the two separate runs of the experiment, the 10″ section of core was placed on an incline apparatus inclined at a approximately 45 degrees and positioned perpendicular to the direction of incline. In each run of the experiment, a burette was mounted approximately 3″ above the center of the section of the core. The burette was adjusted for a flow rate of 30 ml/minute and was filed with approximately 30 ml's of fluid. A piece of absorbent material having a relatively insignificant weight was placed at the mouth of the incline apparatus to catch run off. After the 30 ml's of fluid was fully discharged to the section of core, the absorbent material at the mouth of the incline apparatus was weighed. This experiment was repeated through 5 total iterations with each core type. The results of the experiment were as follows:

TABLE 1.1

| | Current | C-Folded |
|---|---|---|
| Sample | Core* | Core* |
| 1 | 9.0 | 0.0 |
| 2 | 6.9 | 0.0 |
| 3 | 7.0 | 0.0 |
| 4 | 7.3 | 0.0 |
| 5 | 8.3 | 0.0 |
| Avg. | 7.7 | 0.0 |

*weight shown in grams

The experiment revealed that under the test conditions, there was virtually no leakage from the section of core having the C-folded fluid barrier, while the conventional absorbent core leaked an average of 7.7 grams of fluid. Therefore, the absorbent core design of the present invention is presumed to have significantly improved fluid retention properties as compared with conventional absorbent core designs which are devoid of a C-folded fluid barrier covering the bottom, sides and at least a portion of the top of the absorbent core.

Therefore, embodiments of the present invention provide an absorbent core for use with absorbent articles that provides improved fluid retention and reduced core leakage over conventional absorbent core designs and that can be manufactured in a manner that reduces and/or minimizes source material costs and design complexity.

While the foregoing description includes many details and specificities, it is to be understood that these have been included for purposes of explanation only, and are not to be interpreted as limitations. Many modifications and equivalent substitutions to the embodiments described above can be made without departing from the spirit and scope of the invention.

What is claimed is:

1. An absorbent article comprising:
a back sheet layer;
a top sheet layer;
an absorbent core comprising synthetic fibers disposed at least partially between the back sheet layer and the top sheet layer;
a C-shaped fluid impervious layer that surrounds at least the back sheet facing surface of the absorbent core and side surfaces of the core; and
a fluid pervious layer that covers at least a portion of the top sheet layer facing surface of the core.

2. The absorbent article according to claim 1, wherein the synthetic fibers are selected from the group consisting of cellulose acetate fibers, polypropylene fibers, polyethylene fibers, polyester fibers, rayon fibers, polyacrylonitrile fibers and mixtures thereof.

3. The absorbent article according to claim 1, wherein the absorbent core comprises a super absorbent polymer (SAP) material.

4. The absorbent article according to claim 1, wherein the fluid impervious layer comprises a fluid impervious film surrounding at least the back sheet facing surface and side surfaces of the absorbent core.

5. The absorbent article according to claim 1, further comprising a fluid acquisition layer disposed between the absorbent core and the top sheet layer.

6. The absorbent article according to claim 5, wherein the fluid acquisition layer comprises an apertured film layer.

7. The absorbent article according to claim 5, wherein the fluid acquisition layer comprises a non-woven acquisition layer.

8. The absorbent article according to claim 5, wherein the fluid acquisition layer covers only the top sheet facing surface of the absorbent core.

9. The absorbent article according to claim 5, wherein the fluid acquisition layer covers the top sheet facing surface and portions of the side surfaces of the absorbent core.

10. The absorbent article according to claim 5, wherein the fluid acquisition layer surrounds the entire absorbent core.

11. The absorbent article according to claim 1, further comprising a fluid management layer.

12. The absorbent article according to claim 11, wherein the fluid management layer is disposed between the absorbent core and the back sheet layer.

13. An absorbent core for use with an absorbent article comprising:
a volume of synthetic absorbent material defined by a top body facing surface, a bottom outer facing surface and side surfaces, wherein at least a portion of the top body facing surface comprises a fluid pervious layer and the remaining surfaces comprise a fluid impervious layer.

14. The absorbent core according to claim 13, wherein the synthetic absorbent material comprises a material selected from the group consisting of cellulose acetate fibers, polypropylene fibers, polyester fibers, polyethylene fibers, rayon fibers, polyacrylonitrile fibers and mixtures thereof.

15. The absorbent core according to claim 13, wherein the absorbent material comprises an SAP material.

16. The absorbent core according to claim 13, wherein the fluid impervious layer comprises a fluid impervious film surrounding at least the bottom outer facing surface and side surfaces of the absorbent core

17. The absorbent core according to claim 13, further comprising a fluid acquisition layer disposed above at least a portion of the top body facing surface.

18. The absorbent core according to claim 17, wherein the fluid acquisition layer comprises an apertured film layer.

19. The absorbent core according to claim 17, wherein the fluid acquisition layer comprises a non-woven acquisition layer.

20. The absorbent core according to claim 17, wherein the fluid acquisition layer covers only the top body facing surface of the absorbent core.

21. The absorbent core according to claim 17, wherein the fluid acquisition layer covers the top body facing surface and portions of the side surfaces of the absorbent core.

22. The absorbent core according to claim 17, wherein the fluid acquisition layer surrounds the entire absorbent core.

23. The absorbent core according to claim 13, further comprising a fluid management layer.

24. The absorbent article according to claim 23, wherein the fluid management layer is disposed over the bottom outer facing surface of the core.

25. A method of manufacturing an absorbent article comprising:
providing a top sheet layer;
providing a back sheet layer;
providing an absorbent core comprising synthetic fibers disposed at least partially between the top sheet layer and the back sheet layer as an absorbent article;
providing a fluid permeable layer over at least a portion of the top sheet layer facing surface of the core; and
providing a fluid impermeable layer over at least some of the remaining surfaces of the core, thereby encasing the core in a combination of fluid permeable and fluid impermeable materials.

26. The method according to claim 25, wherein the step of providing an absorbent core of synthetic fibers comprises providing an absorbent core of a material selected from the group consisting of cellulose acetate fibers, polypropylene fibers, polyethylene fibers, polyester fibers, rayon fibers, polyacrylonitrile fibers and mixtures thereof.

27. The method according to claim 25, where the step of providing an absorbent core of synthetic fibers comprises providing an absorbent core having an SAP material.

28. The method according to claim 25, further comprising providing a fluid acquisition layer between the top sheet layer and the absorbent core.

29. The method according to claim 28, the step of providing a fluid acquisition layer comprising providing an apertured film layer.

30. The method according to claim 28, the step of providing a fluid acquisition layer comprising providing a non-woven fluid acquisition layer.

31. The method according to claim 28, the step of providing a fluid acquisition layer comprises providing a fluid acquisition layer covering only the top sheet facing surface of the absorbent core.

32. The method according to claim 28, the step of providing a fluid acquisition layer comprises providing a fluid acquisition layer covering the top sheet facing surface and portions of the side surfaces of the absorbent core.

33. The method according to claim 28, the step of providing a fluid acquisition layer comprises providing a fluid acquisition layer covering the entire absorbent core.

34. The method according to claim 25, wherein the step of providing a fluid impermeable layer comprises providing a fluid impervious film surrounding at least the back sheet facing surface and side surfaces of the absorbent core.

35. The method according to claim 25, further comprising providing a fluid management layer.

36. The method according to claim 25, further comprising providing a fluid management layer over at least the back sheet facing surface of the core.

37. An absorbent article having a C-folded absorbent core comprising:
a back sheet layer;
a top sheet layer;
an absorbent core comprising synthetic fibers at least partially disposed between at least portions of the top sheet layer and the back sheet layer;
a fluid pervious layer covering at least a portion of the surface of the absorbent core facing the top sheet layer; and
a fluid impervious layer covering at least some of the remaining surfaces of the absorbent core, wherein the fluid impervious layer comprises a C-like cross sectional shape surrounding the absorbent core.

38. The absorbent article according to claim 37, wherein the absorbent core comprises a synthetic fiber selected from the group consisting of cellulose acetate fibers, polypropylene fibers, polyethylene fibers, polyester fibers, rayon fibers, polyacrylonitrile fibers and mixtures thereof.

39. The absorbent article according to claim 37, wherein the absorbent core comprises a super absorbent polymer (SAP) material.

40. The absorbent article according to claim 37, wherein the fluid impervious layer comprises a fluid impervious film surrounding at least the back sheet facing surface and side surfaces of the absorbent core.

41. The absorbent article according to claim 37, further comprising a fluid acquisition layer disposed between the absorbent core and the top sheet layer.

42. The absorbent article according to claim 41, wherein the fluid acquisition layer comprises an apertured film layer.

43. The absorbent article according to claim 41, wherein the fluid acquisition layer comprises a non-woven acquisition layer.

44. The absorbent article according to claim 41, wherein the fluid acquisition layer covers only the top sheet facing surface of the absorbent core.

45. The absorbent article according to claim 41, wherein the fluid acquisition layer covers the top sheet facing surface and portions of the side surfaces of the absorbent core.

46. The absorbent article according to claim 41, wherein the fluid acquisition layer surrounds the entire absorbent core.

47. The absorbent article according to claim 37, further comprising a fluid management layer.

48. The absorbent article according to claim 47, wherein the fluid management layer is disposed between the absorbent core and the back sheet layer.
